(19) United States
(12) Patent Application Publication
(10) Pub. No.: US 2006/0228419 A1
(43) Pub. Date: Oct. 12, 2006

(54) Solvent-free process for preparation of hydrophilic dispersions of nanoparticles of inclusion complexes

(75) Inventors: Rina Goldshtein, Har Hebron (IL); Larisa Gitis, Holon (IL); Vladimir Mikunis, Raanana (IL); Galina Ratner, Rehovot (IL); Boris Tulbovich, Ashkelon (IL); Ilya Zelkind, Ofakim (IL); Vadim Goldshtein, Har Hebron (IL)

Correspondence Address: BROWDY AND NEIMARK, P.L.L.C.;624 NINTH STREET, NW, SUITE 300, WASHINGTON, DC, 20001-5303, US

(73) Assignee: SoluBest Ltd., Rehovot (IL)

(21) Appl. No.: 11/100,620

(22) Filed: Apr. 7, 2005

(51) Int. Cl. A61K 31/7048; A61K 31/7052; A61K 9/14

(52) U.S. Cl. 424/489; 514/28; 514/29; 977/906

(57) ABSTRACT

The invention provides a solvent-free process for the preparation of a hydrophilic dispersion comprising nanoparticles of an hydrophilic inclusion complex consisting essentially of nanosized particles of an active compound and an amphiphilic polymer which wraps said active compound such that non-valent bonds are formed between said compound and said polymer in said inclusion complex, comprising: (i) preparation of an aqueous solution of the amphiphilic polymer; and (ii) bringing the active compound and the polymer aqueous solution into interaction under conditions suitable for the formation of said hydrophilic dispersion. The process can be applied to small organic compounds as well as to macromolecules, and provides stable hydrophilic dispersions.

### FIELD OF THE INVENTION

The present invention is in the field of nanoparticles. More particularly, the invention relates to an organic solvent-free process for the preparation of soluble nanosized particles consisting of inclusion complexes of an active molecule wrapped within a suitable amphiphilic polymer.

### BACKGROUND OF THE INVENTION

Two formidable barriers to effective drug delivery and hence to disease treatment, are solubility and stability. To be absorbed in the human body, a compound has to be soluble in both water and fats (lipids). Solubility in water is, however, often associated with poor fat solubility and vice-versa.

Over one third of drugs listed in the U.S. Pharmacopoeia and about 50% of new chemical entities (NCEs) are insoluble or poorly insoluble in water. Over 40% of drug molecules and drug compounds are insoluble in the human body. In spite of this, lipophilic drug substances having low water solubility are a growing drug class having increasing applicability in a variety of therapeutic areas and for a variety of pathologies.

Solubility and stability issues are major formulation obstacles hindering the development of therapeutic agents. Aqueous solubility is a necessary but frequently elusive property for formulations of the complex organic structures found in pharmaceuticals. Traditional formulation systems for very insoluble drugs have involved a combination of organic solvents, surfactants and extreme pH conditions. These formulations are often irritating to the patient and may cause adverse reactions.

The size of the drug molecules also plays a major role in their solubility and stability as well as bioavailability. Bioavailability refers to the degree to which a drug becomes available to the target tissue or any alternative in vivo target (i.e., receptors, tumors, etc.) after being administered to the body. Poor bioavailability is a significant problem encountered in the development of pharmaceutical compositions, particularly those containing an active ingredient that is poorly soluble in water. Poorly water-soluble drugs tend to be eliminated from the gastrointestinal tract before being absorbed into the circulation. It is known that the rate of dissolution of a particulate drug can increase with increasing surface area, that is, decreasing particle size

Recently, there has been an explosion of interest in nanotechnology, the manipulation on the nanoscale. Nanotechnology is not an entirely new field: colloidal sols and supported platinum catalysts are nanoparticles. Nevertheless, the recent interest in the nanoscale has produced, among numerous other things, materials used for and in drug delivery. Nanoparticles are generally considered to be solids whose diameter varies between 1-1000 nm.

Although a number of solubilization technologies do exist, such as liposomes, cylcodextrins, microencapuslation, and dendrimers, each of these technologies has a number of significant disadvantages.

Liposomes, as drug carriers, have several potential advantages, including the ability to carry a significant amount of drug, relative ease of preparation, and low toxicity if natural lipids are used. However, common problems encountered with liposomes include: low stability, short shelf-life, poor tissue specificity, and toxicity with non-native lipids. Additionally, the uptake by phagocytic cells reduces circulation times. Furthermore, preparing liposome formulations that exhibit narrow size distribution has been a formidable challenge under demanding conditions, as well as a costly one. Also, membrane clogging often results during the production of larger volumes required for pharmaceutical production of a particular drug.

Cyclodextrins are crystalline, water-soluble, cyclic, non-reducing oligo-saccharides built from six, seven, or eight glucopyranose units, referred to as alpha, beta and gamma cyclodextrin, respectively, which have long been known as products that are capable of forming inclusion complexes. The cyclodextrin structure provides a molecule shaped like a segment of a hollow cone with an exterior hydrophilic surface and interior hydrophobic cavity. The hydrophilic surface generates good water solubility for the cyclodextrin and the hydrophobic cavity provides a favorable environment in which to enclose, envelope or entrap the drug molecule. This association isolates the drug from the aqueous solvent and may increase the drug's water solubility and stability.

For a long time, most cyclodextrins had been no more than scientific curiosities due to their limited availability and high price, but lately cyclodextrins and their chemically modified derivatives became available commercially, generating a new technology of packing on the molecular level. Cyclodextrins are, however, fraught with disadvantages including limited space available for the active molecule to be entrapped inside the core, lack of pure stability of the complex, limited availability in the marketplace, and high price.

Microencapsulation is a process by which tiny parcels of a gas, liquid, or solid active ingredient (“core material”) are packaged within a second material for the purpose of shielding the active ingredient from the surrounding environment. These capsules, which range in size from one micron (one-thousandth of a millimeter) to approximately seven millimeters, release their contents at a later time by means appropriate to the application.

There are four typical mechanisms by which the core material is released from a microcapsule: (1) mechanical rupture of the capsule wall, (2) dissolution of the wall, (3) melting of the wall, and (4) diffusion through the wall. Less common release mechanisms include ablation (slow erosion of the shell) and biodegradation.

Microencapsulation covers several technologies, where a certain material is coated to obtain a micro-package of the active compound. The coating is performed to stabilize the material, for taste masking, preparing free flowing material of otherwise clogging agents etc. and many other purposes. This technology has been successfully applied in the feed additive industry and to agriculture. The relatively high production cost needed for many of the formulations is, however, a significant disadvantage.

In the cases of nanoencapsulation and nanoparticles (which are advantageously shaped as spheres and, hence, nanospheres), two types of systems having different inner structures are possible: (i) a matrix-type system composed of an entanglement of oligomer or polymer units, defined as nanoparticles or nanospheres, and (ii) a reservoir-type system, consisting of an oily core surrounded by a polymer wall, defined as a nanocapsule.

Depending upon the nature of the materials used to prepare the nanospheres, the following classification exists: (a) amphiphilic macromolecules that undergo a cross-linking reaction during preparation of the nanospheres; (b) monomers that polymerize during preparation of the nanoparticles; and (c) hydrophobic polymers, which are initially dissolved in organic solvents and then precipitated under controlled conditions to produce nanoparticles.

Problems associated with the use of polymers in micro- and nanoencapsulation include the use of toxic emulgators in emulsions or dispersions, polymerization or the application of high shear forces during emulsification process, insufficient biocompatibility and biodegradability, balance of hydrophilic and hydrophobic moieties, etc. These characteristics lead to insufficient drug release.

Dendrimers are a class of polymers distinguished by their highly branched, tree-like structures. They are synthesized in an iterative fashion from ABn monomers, with each iteration adding a layer or “generation” to the growing polymer. Dendrimers of up to ten generations have been synthesized with molecular weights in excess of 106 kDa. One important feature of dendrimeric polymers is their narrow molecular weight distributions. Indeed, depending on the synthetic strategy used, dendrimers with molecular weights in excess of 20 kDa can be made as single compounds.

Dendrimers, like liposomes, display the property of encapsulation, and are able to sequester molecules within the interior spaces. Because they are single molecules, not assemblies, drug-dendrimer complexes are expected to be significantly more stable than liposomal drugs. Dendrimers are thus considered as one of the most promising vehicles for drug delivery systems. However, the dendrimer technology is still in the research stage, and it is speculated that it will take years before it is applied in the industry as an efficient drug delivery system.

U.S. patent applications Ser. No. 10/952,380, Ser. No. 10/256,023 (Publication US 2003/0129239), and Ser. No. 09/966,847 (Publication US 2003/0064924), assigned to the same assignee of this application, and incorporated herewith by reference in their entirety as if fully disclosed herein, disclose a novel technology, called by the applicants “Solumer technology”, for preparing nanosized inclusion complexes of active compounds. The process described in these applications is a bi-phase system where both aqueous and organic solvents are used, wherein the latter is eliminated upon termination of the Solumerization (formation of water-soluble nanoparticles) process.

Processes which are not dependent on organic solvents are often considered more user friendly than those that employ these solvents. Especially for the pharmaceutical industry and oftentimes for the chemical industry as well, environmental and health regulations often limit the use of inactive process components. Even when not limited by specific regulations prohibiting their use, the elimination of organic solvents or carrying out processes in their absence results in significantly more cost effective processes.

Reference is made to copending applications to be assigned to the same assignee entitled “Hydrophilic dispersions of nanoparticles of inclusion complexes of macromolecules” and “Hydrophilic dispersions of nanoparticles of inclusion complexes of salicylic acid”, filed on the same date at the United States Patent and Trademark Office.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

It has now been found in accordance with the present invention that the solumerization technology disclosed in the above-mentioned U.S. application Ser. No. 10/952,380, Ser. No. 10/256,023, and Ser. No. 09/966,847, can be performed for small organic compound as well as for macromolecules without the use of organic solvents.

The present invention thus relates to a solvent-free process for the preparation of a hydrophilic dispersion comprising nanoparticles of an hydrophilic inclusion complex consisting essentially of nanosized particles of an active compound and an amphiphilic polymer which wraps said active compound such that non-valent bonds are formed between said compound and said polymer in said inclusion complex, said process comprising:

(i) preparing an aqueous solution of the amphiphilic polymer; and

(ii) bringing the active compound and the polymer aqueous solution into interaction under conditions suitable for the formation of said hydrophilic dispersion.

### BRIEF DESCRIPTION OF THE DRAWINGS

FIG. 1 depicts the light scattering measurements of the sizes of nanoparticles of salicylic acid, that were prepared by the one-step solvent-free method using 0.2% polyacrylamide (PAA) modified with 2.1% urea, as described in Example 3. The analysis was performed using a Zetasizer Nano light scattering technique (a resolution of 0.6-6000 nm) with a 1:10 dilution of the samples.

FIG. 2 is a photograph of a cryo-transmission electron microscopy (TEM) analysis, showing nanoparticles of salicylic acid that were prepared by the two-step solvent-free method using 0.2% PAA modified with 2.2% urea, as described in Example 2.

FIGS. 3A-3D show the Fourier transform infrared (FTIR) spectroscopy analysis of pure salicylic acid (FIG. 3A), sodium salicylate salt (FIG. 3B) and nanoparticles of inclusion complexes of salicylic acid (6.23% and 6.48%) which were prepared by the two-step and one-step organic solvent-free processes, respectively (FIGS. 3C and 3D, respectively).

FIG. 4 is a graph showing the release of salicylic acid from inclusion complexes prepared using the two-step organic solvent-free process described in Example 2 herein, following a dialysis. Dialysis was performed using dialysis tubing having a pore size of either 3500 Daltons (MW 3500) or 7000 Daltons (MW 7000), filled with 2 ml of a dispersion comprising nanoparticles of salicylic acid having a final salicylic acid concentration of 7%. At the indicated times, 1 ml aliquots were removed from the external solution for analysis of the salicylic acid content by reverse-phase high-pressure liquid chromatography (RP-HPLC). Measurement of the salicylic acid in each experimental sample was followed by calculation of the salicylic acid concentration according to the area of salicylic acid peak in that sample.

FIG. 5 is a graph showing the stability of the inclusion complex of 0.1% sodium hyaluronate (NaHA) with 1.9% hydrolyzed potato starch (HPS) or with 1.9% modified corn starch B-790 (B-790) in the presence of hyaluronidase, measured after 3 and 5 hours.

FIG. 6 is a graph showing the stability of the inclusion complex of 0.5% sodium hyaluronate (NaHA) with 1.9% hydrolyzed potato starch (HPS) or with 1.9% modified corn starch B-790 (B-790) in the presence of of hyaluronidase, measured after 3 and 5 hours.

### DETAILED DESCRIPTION OF THE INVENTION

The present invention provides a process for the production of soluble nanoparticles and, in particular, an organic solvent-free process for the preparation of hydrophilic dispersions of nanoparticles of inclusion complexes of an active compound in amphiphilic polymers.

The soluble nanoparticles prepared by the process of the invention are referred to herein sometimes as “solu-nanoparticles” or “solumers”. They are differentiated by the use of water-soluble amphiphilic polymers that are capable of producing molecular complexes with active compounds, particularly pharmaceutical drugs. The solunanoparticles formed in accordance with the present invention render water-insoluble active compounds soluble in water and readily bioavailable in the human body.

As used herein, the term “inclusion complex” refers to a complex in which one component—the amphiphilic polymer (the “host), forms a cavity in which molecular entities of a second chemical species—the active compound (the “guest”), are located. Thus, in accordance with the present invention, inclusion complexes are produced in which the host is the amphiphilic polymer and the guest is the active compound wrapped and fixated or secured within the cavity or space formed by said amphiphilic polymer host.

The inclusion complexes produced in accordance with the present invention contain the active compound, which interacts with the amphiphilic polymer by non-valent interactions and form a polymer-active compound as a distinct molecular entity. A significant advantage and unique feature of the inclusion complex of the present invention is that no new chemical bonds are formed and no existing bonds are destroyed during the formation of the inclusion complex (very important for pharmaceutical drugs). The particles comprising the inclusion complexes are nanosized and no change occurs in the active compound molecule itself, when it is enveloped, or advantageously wrapped, by the polymer.

Another important characteristic of the inclusion complexes produced by the process of the invention is that the active compound may be presented in a non-crystalline state. As used herein, the term “non-crystalline” state is intended to include both disordered crystalline and, preferably, amorphous state. Thus, in preferred embodiments, the active compound is in amorphous form. It is known in the art that the amorphous state is preferred for drug delivery as it may indeed enhance bioavailability.

The creation of the complex does not involve the formation of any valent bonds (which may change the characteristics or properties of the active macromolecular compound). As used herein, the term “non-valent” is intended to refer to non-covalent, non-ionic and non-semi-polar bonds and/or interactions, and includes weak, non-covalent bonds and/or interactions such as electrostatic forces, Van der Waals forces, and hydrogen bonds formed during the creation of the inclusion complex. The formation of non-valent bonds preserves the structure and properties of the active compound.

The solunanoparticles produced by the process of the invention remain stable for long periods of time, may be manufactured at a low cost, and may improve the overall bioavailability of the active compound.

The active compound may be a small molecule as described in the above-mentioned U.S. patent applications Ser. No. 10/952,380, Ser. No. 10/256,023 and Ser. No. 09/966,847, and in the copending application entitled “Hydrophilic dispersions of nanoparticles of inclusion complexes of salicylic acid”, filed on the same date at the United States Patent and Trademark Office, or the active compound may be a macromolecule, as described in the copending application entitled “Hydrophilic dispersions of nanoparticles of inclusion complexes of macromolecules”, filed on the same date at the United States Patent and Trademark Office, each and all of these applications being incorporated herewith by reference in their entirety as if fully disclosed herein.

The present invention provides a solvent-free process for forming nanoparticles of inclusion complexes of an active compound wrapped in an amphiphilic polymer, which uses only aqueous solutions. In accordance with the present invention, this concept has been extended to several pharmaceutical active compounds and is noted amongst the examples given herein. The solvent-free process is possible with water-soluble active compounds as well as for partially or full water-insoluble compounds when appropriate general conditions can be found, such as for example, pH, temperature, pressure, and the like, in which the water-insoluble active compound is soluble in water under these conditions, thus allowing the nanosized particles of the inclusion complex to form before these conditions revert back to those standard ones that will not allow for solubility of the active compound. An example of conditions for solubility in water of a water-insoluble compound is the case of the macrolide antibiotic azithromycin, that can be dissolved in an aqueous solution of acidic pH, e.g. pH of about 3-5. As shown herein in Example 11, chitosan was first added to an acid solution (acetic acid) and had a pH of 4.0-4.4 when azithromycin was added to the polymer solution. Under these conditions, the azithromycin was soluble in the polymer solution and could form the desired water-soluble solumer.

The nanoparticles produced by the process of the present invention comprise the active compound or core wrapped within a water-soluble amphiphilic polymer. As described in the parent U.S. applications Ser. No. 10/256,023 and Ser. No. 09/966,847, hereby incorporated by reference in their entirety, a variety of different polymers can be used for any of the selected active compounds. The polymer used in the formation of the nanosoluparticles are selected according to an algorithm that takes into account various physical properties of the active compounds and the polymer or polymers, as well as their future interaction in the resulting complex. The algorithm is utilized in this manner to select the optimal polymer(s) and takes into consideration the following properties of the polymer itself in selecting a polymer for the active molecule/polymer interaction in the formation of the complex: molecular weight, basic polymer chain length, the length of the kinetic unit, the solubility of the polymer in water, the overall degree of solubility, the degree of polymer flexibility, the hydrophilic-lipophilic balance (HLB), and the polarity of the hydrophilic groups of the polymer. The main properties of the polymer include its HLB, the length and the flexibility of its polymer chain, and also the state of polarity of the hydrophilic groups.

Thus, one important parameter in the choice of the polymer or polymers is the HLB, i.e., the measure of the molecular balance of the hydrophilic and lipophilic portions of the compound. Within the HLB International Scale of 0-20, lipophilic molecules have a HLB of less than 6, and hydrophilic molecules have a HLB of more than 6. Thus, according to the present invention, the HLB of the polymer is selected in such a way that, after combining to it the active compound, the total resulting HLB value of the complex will be greater than 8, rendering the complex water-soluble.

The solvent-free process of the present invention comprises the steps of:

(i) preparation of an aqueous solution of the amphiphilic polymer; and

(ii) bringing the active compound and the polymer aqueous solution into interaction under conditions suitable for the formation of the hydrophilic dispersion comprising nanoparticles of an hydrophilic inclusion complex consisting essentially of nanosized particles of an active compound and an amphiphilic polymer which wraps said active compound such that non-valent bonds are formed between said compound and said polymer in said inclusion complex.

In one embodiment, in step (ii), the active compound is added as a powder to the polymer aqueous solution. Examples of this embodiment are the preparation of solumers of salicylic acid and azithromycin as disclosed herein.

In another embodiment, in step (ii), an aqueous solution of the active compound is added dropwise to the polymer aqueous solution under constant mixing. Examples of this embodiment are the preparation of solumers of macromolecules as disclosed herein.

In one embodiment, the invention provides a process for the preparation of a hydrophilic dispersion comprising nanoparticles of inclusion complexes of salicylic acid wrapped in an amphiphilic polymer such that non-valent bonds are formed between the salicylic acid and the amphiphilic polymer, wherein said amphiphilic polymer is selected from the group consisting of polyacrylic acid, polyacrylamide and copolymers thereof, polymethacrylamide and copolymers thereof, and polylysine, and said polymer is modified by reaction with urea or a derivative thereof, nicotinamide or guanidine. The amphiphilic polymer may be a copolymer of acrylamide or methacrylamide with one or two monomers selected from the group consisting of acrylic acid, methacrylic acid, an alkyl acrylate, an alkyl methacrylate, acrylonitrile, ethyleneimine, vinyl acetate, styrene, maleic anhydride and vinyl pyrrolidone. The modifier may be a urea derivative selected from the group consisting of methylol urea, acetyl urea, semicarbazide and thiosemicarbazide. In a preferred embodiment of the invention, the amphiphilic polymer is polyacrylamide modified by reaction with urea.

The solvent-free process for the preparation of the hydrophilic dispersion comprising nanoparticles of inclusion complexes of salicylic acid may be a two-step process, comprising:

(i) preparation of a solution of the amphiphilic polymer in water;

(ii) modification of the amphiphilic polymer by reaction with urea or a derivative thereof, nicotinamide or guanidine, under heat and pressure, in an autoclave;

(iii) addition of salicylic acid powder to the modified polymer water solution; and

(iv) subjecting the dispersion obtained in (iii) to autoclave treatment, thus obtaining the desired dispersion comprising nanoparticles of inclusion complexes of salicylic acid entrapped within said modified amphiphilic polymer.

In another embodiment, the invention relates to a one-step solvent-free process for the preparation of the hydrophilic dispersion comprising nanoparticles of inclusion complexes of salicylic acid, comprising:

(i) preparation of a solution of the amphiphilic polymer in water;

(ii) modification of the amphiphilic polymer by reaction with urea or a derivative thereof, nicotinamide or guanidine;

(iii) addition of salicylic acid powder to the modified polymer water solution; and

(iv) subjecting the dispersion obtained in (iii) to autoclave treatment, thus obtaining the desired hydrophilic dispersion comprising nanoparticles of inclusion complexes of salicylic acid entrapped within said amphiphilic polymer.

In another embodiment of the present invention, the active compound is a macromolecule such as a polypeptide of molecular weight above 1,000 Da, a protein, a nucleic acid or a polysaccharide, and the amphiphilic polymer is a polysaccharide, in natural form or modified. The polysaccharide may be starch, chitosan or an alginate.

For use in the preparation of the inclusion complexes of macromolecules, it is desirable to use starch with a large proportion of linear chains, i.e. starch with high contents of amylose, the constituent of starch in which anhydroglucose units are linked by a-D-1,4 glucosidic bonds to form linear chains, and low contents of amylopectin, a constituent of starch having a polymeric, branched structure. The levels of amylose and amylopectin and their molecular weight vary between different starch types. Encompassed by the present invention are starches of various sources such as potato, maize/corn, wheat, and tapioca/cassava starch.

To improve its characteristics for use in the invention, starch, e.g. corn or potato starch, can be modified, for example by increasing its hydrophilicity by acid hydrolysis and/or by reaction with an agent, e.g. polyethylene glycol (PEG) and/or hydrogen peroxide. In addition, pregelatinized starch can be used as well as starch subjected to thermal treatment to reduce the amount of branching (designated “thermodestructed starch”).

According to the process of the invention, dispersions of inclusion complexes of a protein (bovine serum albumin), an enzyme (hyaluronidase) and a polysaccharide (hyaluronic acid in the form of its sodium salt) have been prepared.

The present invention thus provides a solvent-free process for preparation of a hydrophilic dispersion comprising nanoparticles of inclusion complexes of an active macromolecule and an amphiphilic polysaccharide which wraps the active macromolecule such that non-valent bonds are formed between said active macromolecule and said amphiphilic polysaccharide, the process comprising the steps of:

(i) preparing a solution of the amphiphilic polysaccharide in water;

(ii) preparing a molecular solution of the active macromolecule in water and

(iii) adding dropwise the water solution of the active macromolecule (ii) into the water polysaccharide solution (i) under constant mixing;

thus obtaining the hydrophilic dispersion comprising nanoparticles of inclusion complexes of said active macromolecule wrapped within said amphiphilic polysaccharide.

In step (ii), the macromolecule aqueous solution is treated with a salt, for example, ammonium sulfate, KCl or NaCl, before addition to the polymer water solution. In step (iii), the macromolecule is added to the warmed polysaccharide solution, when the macromolecule is not a protein, as shown herein for hyaluronic acid. When the macromolecule is a protein, as shown herein for bovine serum albumin and hyaluronidase, the macromolecule is added to the polysaccharide solution at room temperature.

In yet a further embodiment of the invention, the active compound is a small organic compound, for example a macrolide antibiotic such as erythromycin, clarithromycin an azithromycin, and the amphiphilic polymer is a polysaccharide such as starch, chitosan or an alginate, natural or modified in order to increase its hydrophilicity or to reduce its branching, or both. Chitosan and alginate may be modified as described above for starch. For the dissolution of the macrolide antibiotic, the polymer solution is first acidified before addition of the antibiotic powder.

The present invention thus further provides a solvent-free process for the preparation of a hydrophilic dispersion comprising nanoparticles of an hydrophilic inclusion complex consisting essentially of nanosized particles of a macrolide antibiotic and an amphiphilic polysaccharide polymer which wraps said active compound such that non-valent bonds are formed between said macrolide antibiotic and said polysaccharide in said inclusion complex, comprising:

(i) preparation of an acidic aqueous solution of the amphiphilic polysaccharide;

(ii) adding the macrolide antibiotic powder to the acidic aqueous polymer solution of (i) under heating and mixing for dissolution of the macrolide antibiotic;

(iii) streaming the solution of (ii) simultaneously with a large volume of water to a high turbulent zone of a vessel for the interaction of the macrolide antibiotic with the polysaccharide; and

(iv) concentrating the aqueous solution of (iii) under constant mixing and heating, thus obtaining the desired dispersion of nanoparticles of inclusion complexes of the macrolide antibiotic wrapped in the polysaccharide.

In one preferred embodiment, the macrolide antibiotic is azithromycin and the polysaccharide is chitosan. The azithromycyn is added in (ii) to the acidified aqueous solution of chitosan (pH=4.0-4.4) to obtain a 1% solution (w/v) of azithromycin in the chitosan aqueous solution. The large volume of water in step (iii) is about 3-4 times larger than the azithromycin/chitosan solution volume and, thus, the water in step (iii) should be streamed faster than the azithromycin/chitosan solution. The high turbulence zone (above 10,000 rpm) in step (iii) is achieved with a homogenizer. In step (iv), the concentration may be carried out in a vessel until the original volume of the azithromycin/chitosan solution of (ii) is achieved.

The dispersions produced by the process of the invention are stable. Stability of the nanoparticles and of the inclusion complexes has more than one meaning. The nanoparticles should be stable as part of a nanocomplex over time, while remaining in the dispersion media. The nanodispersions are stable over time without separation of phases. Furthermore, the non-crystalline or amorphous state should be also retained over time.

It is worth noting that in the process used in the present invention, the components of the system do not result in micelles nor do they form classical dispersion systems. The technology of the present invention causes the following:

(i) after dispersion of the active compound to nanosized particles and fixation by the polymer to form an inclusion complex, enhanced solubility in physiological fluids, in vivo improved absorption, and improved biological activity, as well as transmission to a stable non-crystalline, preferably amorphous, state, are achieved;

(ii) a otherwise crystalline biologically-active molecule becomes non-crystalline, e.g., amorphous, and thus exhibits improved biological activity.

In most preferred embodiments of the present invention, not less than 80% of the nanoparticles in the nanodispersion are within the size range, when the size deviation is not greater than 20%, and the particle size is within the nano range, namely less than 1000 nm, more preferably 100 nm or less.

In an advantageous and preferred embodiment of the invention, the amphiphilic polymer “wraps” the active compound via non-valent interactions. The non-valent bonds or interactions such as electrostatic forces, Van der Waals forces, and hydrogen bonds formed between the polymer and the active compound in the inclusion complex fixate the active compound within the polymer, thus reducing its molecular mobility. The formation of any valent bonds could change the characteristics or properties of the active compound. The formation of non-valent bonds preserves the structure and properties of the active compound, which is particularly important when the active compound is a pharmaceutical.

The aqueous nanodispersions produced by the process of the invention can be lyophilized and then mixed with pharmaceutically acceptable carriers to provide stable pharmaceutical compositions.

The invention will now be illustrated by the following non-limiting examples.

### EXAMPLES

### Example 1

### Preparation of Polymer-Urea-Modified Polyacrylamide (PAA)

Various conditions were used for preparing the polymer solutions of polyacrylamide (PAA) and urea-modified-PAA, as shown in Table 1. The unmodified PAA was used in some experiments but did not provide good results.

Specifically, 3.3 (or 1) grams of PAA (CAS Number 9003058; Acros Organic, New Jersey, USA) were dissolved per liter water by thorough stirring, while heating at 60-90° C. (preferably 80-90° C.) for 80-120 min. Then, 2.1-4 grams of urea were dissolved per liter of the resulting solution by thorough stirring. The mixture was heated to above 100° C. (110-125° C.) under pressure (up to 2 atm) in an autoclave for about 80 minutes, in order to complete the reaction between the polymer and the urea. The resulting pH and viscosity of this and the other solutions prepared by variations on this process were measured and are presented in Table 1. The solution was cooled to room temperature before proceeding to the step in which the inclusion complexes are formed. Regardless of the amount of urea added within the range noted above and of PAA heating time, the quality of the resulting polymeric solution was generally robust as long as urea treatment was done. These conditions yielded a solution with an average pH of 9.2 (general range between 9.03-9.37) and an average viscosity of 38.4 cP (general range between 29.4-52.8 cP, except in one instance). As expected, 0.1% PAA solutions had lower viscosities than 0.33% PAA solutions. When no urea was added, or when urea was added, but the autoclaving step was omitted, the average pH of the resulting solution was 6.56 (5.98-7.37), and the average viscosity was 16.6 cP (6-29 cP). Therefore, the autoclaving step was considered necessary for preparing urea-modified PAA.

TABLE 1
Polymer preparation for salicylic acid

| Urea concen- | | | | |
|---|---|---|---|---|
| tration for | | | | |
| modification | PAA Heating | Autoclaving | | Viscosity |
| of PAA* (%) | Time | Time | pH | (cP) |
| 2.1 | (No heating) | 80 min | 9.04 | 38.3 |
| 2.2 | 1 hr 23 min | 80 min | 9.31 | 42 |
| 2.2 | 1 hr 23 min | 80 min | 9.26 | 17.5 |
| 2.2 | 1 hr 23 min | 80 min | 9.34 | 29.4 |
| 2.2 | 1 hr 23 min | 80 min | 9.08 | 42.3 |
| 2.2 | 1 hr 03 min | 80 min | 9.17 | 38.9 |
| 2.2 | 1 hr 23 min | 80 min | 9.22 | 52.8 |
| 2.2 | 1 hr 23 min | 80 min | 9.08 | 39.42 |
| 2.2 | 1 hr 23 min | 80 min | 9.16 | 40.8 |
| 2.2 | 1 hr 40 min | 80 min | 9.17 | 36.9 |
| 2.2 | 2 h | 80 min | 9.25 | 39.5 |
| 2.2 | 2 h | 80 min | 9.24 | 38.4 |
| 2.2 | 2 h | 80 min | 9.28 | 38.8 |
| 2.2 | 1 hr 20 min | 80 min | 9.08 | 36 |
| 2.2 | 1 hr 20 min | 80 min | 9.05 | 38.8 |
| 2.2 | (No heating) | 80 min | 9.16 | 37.8 |
| 3 | (No heating) | (No autoclaving) | 5.98 | 6.0 |
| 3 | 1 hr 20 min | 80 min | 9.24 | 32.1 |
| 3 | 1 hr 23 min | 80 min | 9.03 | 40.2 |
| 3 | 1 hr 23 min | 80 min | 9.07 | (Not done) |
| 3 | 1 hr 40 min | (No autoclaving) | 7.37 | 11.1 |
| 4 | 1 hr 20 min | 80 min | 9.23 | 29.4 |
| (none) | 1 hr 20 min | 80 min | 6.93 | 10.5 |
| (none) | (No heating) | (No autoclaving) | 6.06 | 26.6 |
| (none) | (No heating) | (No autoclaving) | 6.32 | 25.47 |
| (none) | (No heating) | (No autoclaving) | 6.09 | 26 |
| (none) | (No heating) | (No autoclaving) | 6.03 | 26 |
| (none) | (No heating) | (No autoclaving) | 6.04 | 29 |
| (none) | 1 hr 30 min | (No autoclaving) | 6.97 | 6.32 |
| (none) | 1 hr 40 min | (No autoclaving) | 6.94 | 6.8 |
| (none) | 1 hr 40 min | 80 min | 6.8 | 9.2 |
| 2.2* | 1 hr 23 min | 80 min | 9.37 | 16.7 |
| 3* | 1 hr 23 min | 80 min | 9.19 | 20.1 |

The PAA concentration was 0.33% except in the instances indicated by an asterisk, when the PAA concentration was 0.1%.

### Example 2

### Two-Step Organic Solvent-Free Process for Preparation of Dispersions Comprising Nanoparticles of Inclusion Complexes of Salicylic Acid Wrapped in Urea-Modified Polyacrylamide

In the two-step process, a solution of PAA and urea in water is prepared as described in Example 1 and autoclaved for about 80 min, and salicylic acid powder is added to the modified polymer solution and autoclaved for about 130-180 min.

The modified polyacrylamide polymer was obtained by reaction of 0.33% or 0.2% PAA with 3% or 2.2% urea. After autoclave, 7.0 grams salicylic acid powder were added for each 100 ml of polymer solution, and the mixture was autoclaved (113-115° C.; 1.50-1.65 atm) for about 130-180 min. The combination of heat and pressure was essential for the solvent-free process, since otherwise significant amounts of crystalline salicylic acid precipitate. Under these conditions, the use of PAA unmodified by urea and of certain polymers such as chitosan or polyvinyl alcohol (PVA) did not lead to the desired dispersions and resulted in the precipitation of salicylic acid

As shown in Table 2, the pH of the resulting dispersion containing the nanoparticles of salicylic acid-polymer complexes ranged 4.46-7.94. Dispersions of salicylic acid with such pHs are suitable for formulations applicable to a variety of routes of administration, including oral, topical, and ocular routes. While the pH of formulations for oral administration is not limited, preparations with a neutral pH are preferred for ocular application and the more acidic preparations are preferred for topical application for skin treatment. The urea concentration was decreased to 2% and the PAA concentration was decreased to 0.18% and the resulting solutions of salicylic acid-polymer complexes had pH values of 4.73 and 4.8 (Table 2, last two rows). Precipitation was found to occur in dispersions having a pH below 4.5. Thus, the combination 0.18-0.2% PAA and 2% urea for the preparation of the modified polymer was found to be more suitable for the preparation of the salicylic acid inclusion complexes for topical use.

The final salicylic acid concentration in the range of 58.52-70.56 mg/ml Table 3) was close to the theoretical (original) concentration value (70 mg/ml).

TABLE 2
Two-step solvent-free preparation of salicylic acid complexes

| | | | pH of the | |
|---|---|---|---|---|
| | | pH of Modified | SA-polymer | SA Conc. |
| % PAA | % Urea* | Polymer | complex | (mg/ml)** |
| 0.33 | 3 | 9.01 | 7.72 | 70.56 |
| 0.33 | 3 | 9.16 | 7.94 | 62.26 |
| 0.2*** | 2.2 | 9.19 | 4.92 | 66.6 |
| 0.2 | 2.2 | 9.18 | 6.46 | 66.82 |
| 0.33 | 2.2 | 8.97 | 6.56 | 69.02 |
| 0.33 | 2.2 | 9.1 | 6.39 | 66.99 |
| 0.2 | 3 | 9.17 | 7.94 | 68.35 |
| 0.2 | 2.2 | 9.19 | 4.92 | 66.6 |
| 0.2 | 2.2 | 9.18 | 6.46 | 66.82 |
| 0.2 | 2.2 | 9.24 | 4.87 | 61.73 |
| 0.2 | 2.2 | 9.24 | 5.16 | 67.01 |
| 0.2 | 2.2 | 9.26 | 4.46 | 58.52 |
| 0.2 | 2.2 | 9.26 | 7.11 | 68.35 |
| 0.18 | 2 | 9.05 | 4.73 | 68.28 |
| 0.18 | 2 | 9.05 | 4.8 | 62.27 |

*concentration of urea (%) for treating PAA
**HPLC measurement of SA in the produced inclusion complexes (mg/ml)
***sample was examined by cryo-TEM

### Example 3

### One-Step Organic Solvent-Free Process for Preparation of Dispersions Comprising Nanoparticles of Inclusion Complexes of Salicylic Acid Wrapped in Urea-Modified Polyacrylamide

In the one-step process, a solution of PAA and urea in water is prepared as described in Example 1, salicylic acid powder is added to the modified polymer solution and autoclaved for about 130-180 min.

The amounts of the reagents and the reaction conditions are similar to the final conditions of Example 2 above. Thus, 0.2 grams polyacrylamide and 2.0 or 2.1 grams of urea were added per 100 ml water, and the mixture was heated to 95° C. while stirring, to form a solution having a pH ranging between 7.42-7.6. Then, approximately 7 grams of salicylic acid powder were added per 100 ml and the mixture was autoclaved (113-115° C.; 1.50-1.65 atm) for about 130-180 min. The complexes were formed during autoclave treatment of the mixture.

The conditions and results are shown in Table 3. No salicylic acid precipitated in these dispersions, as reflected by the low solution turbidity (Y1) and the concentration of salicylic acid in solution within the range 58.94-69.03 mg/ml (Y2). Furthermore, the pH of the final complexes within the range 4.38-5.89 (Y3) was suitable for topical application (about 4.7). The turbidity of the solutions was measured with a SMART2 colorimeter (La Motte Company, Chestertown, Mass., USA) within a scale of 0-400 FTU (formazin turbidity unit). The results for turbidity in Table 3 (Y1: from 0 to 4) are for very clear solutions.

Attempts to shorten the autoclave treatment step by 10 minutes or more resulted in subsequent salicylic acid precipitation.

TABLE 3
Conditions for preparation of salicylic acid (SA)
nanoparticles by the one-step solvent-free method

| Trial | X1 | X2 | pH PAA-U | X3 | Y1 | Y2 | Y3 |
|---|---|---|---|---|---|---|---|
| 1 | 0.2 | 2.1 | 7.58 | 1 hr 50 min | 4 | 61.88 | 4.45 |
| 2 | 0.2 | 2.1 | 7.58 | 2 hr | 1 | 69.03 | 4.38 |
| 3 | 0.2 | 2.1 | 7.58 | 2 hr 10 min | 0 | 61.27 | 4.68 |
| 4* | 0.2 | 2.1 | 7.6 | 2 hr 10 min | 0 | 73.24 | 4.82 |
| 5 | 0.2 | 2.0 | 7.6 | 3 hr | 0 | 58.94 | 5.89 |
| 6 | 0.2 | 2.1 | 7.6 | 2 hr 20 min | 0 | 63.71 | 5.11 |

X1 - Concentration polyacrylamide (PAA)
X2 - Concentration urea (U)
X3 - Time of autoclaving complex
Y1 - Turbidity (FTU, scale 0-400)
Y2 - HPLC analysis (SA mg/ml)
Y3 - pH Complex SA/PAA-U
*Sample Sa-35-87-1

### Example 4

### Physical Analyses of Dispersions Comprising Nanoparticles of Inclusion Complexes of Salicylic Acid

(i) Particle Size Analyses

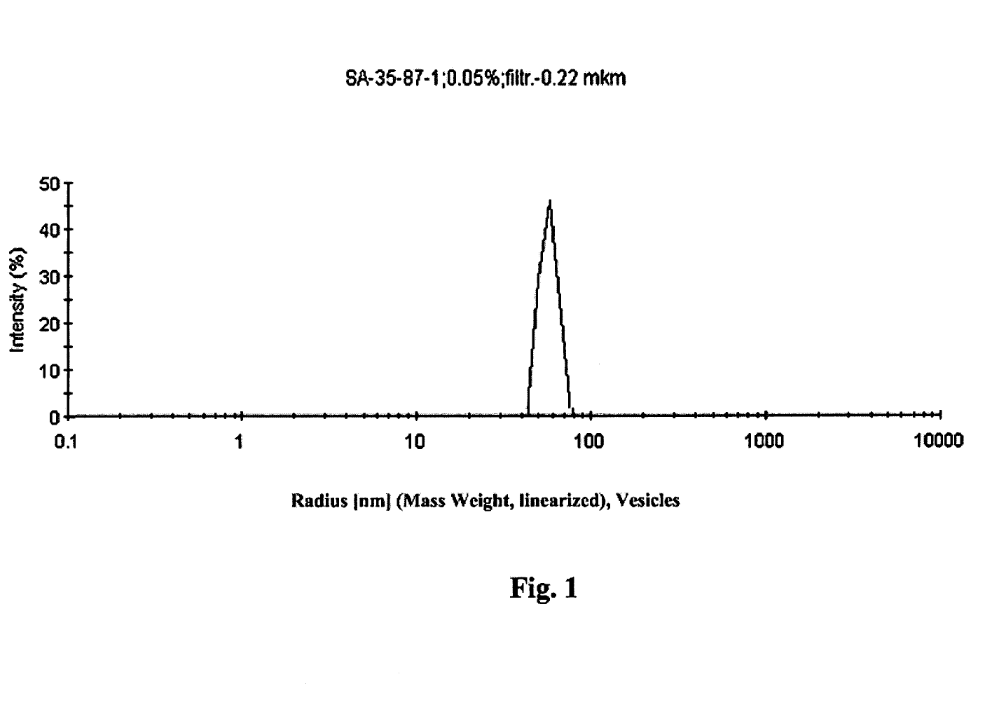

The size of nanoparticles of inclusion complexes of salicylic acid was analyzed using two methods, light scattering and cryo-transmission electron microscopy (TEM). Light scattering measurements of the nanoparticles size were performed using a Zetasizer Nano (Malvern Instruments, Ltd. Worcestershire, United Kingdom), which has a resolution of 0.6-6000 nm. Zetasizer Nano is a dynamic light scattering technique used to estimate the mean particle size. Dispersions comprising nanoparticle prepared as described in Example 3 were measured using this method. A 1:10 dilution of the samples was found necessary for sample analysis. A typical graph of the particle size distribution, depicted in FIG. 1 (sample SA-35-87-1, of Table 3, Trial 4), shows that the diameters of the particles in the dispersions are typically about 50 nm. The narrow peaks obtained by these measurements indicate the high uniformity of the nanoparticle sizes in the dispersions.

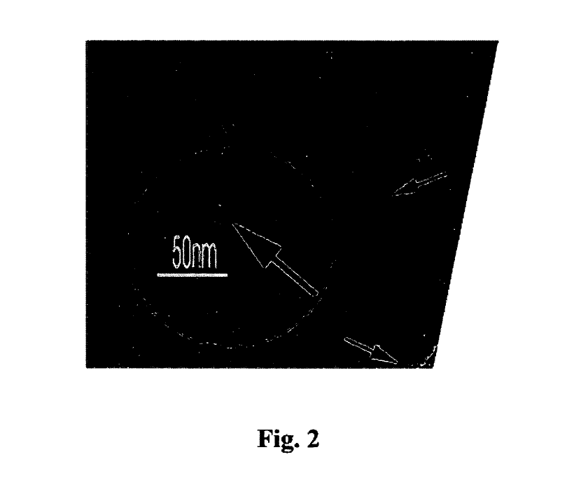

Cryo-TEM was also used to measure the size of the nanoparticles of inclusion complexes of salicylic acid. A sample prepared in Example 2 (as identified in Table 2) was examined by this method and the result shown in FIG. 2 demonstrates that the diameter of the salicylic acid nanoparticles is typically smaller than 50 nm. Therefore, both the one-step and two-step solvent-free methods yield dispersions having nanoparticles with similar sizes.

(ii) FTIR Analyses

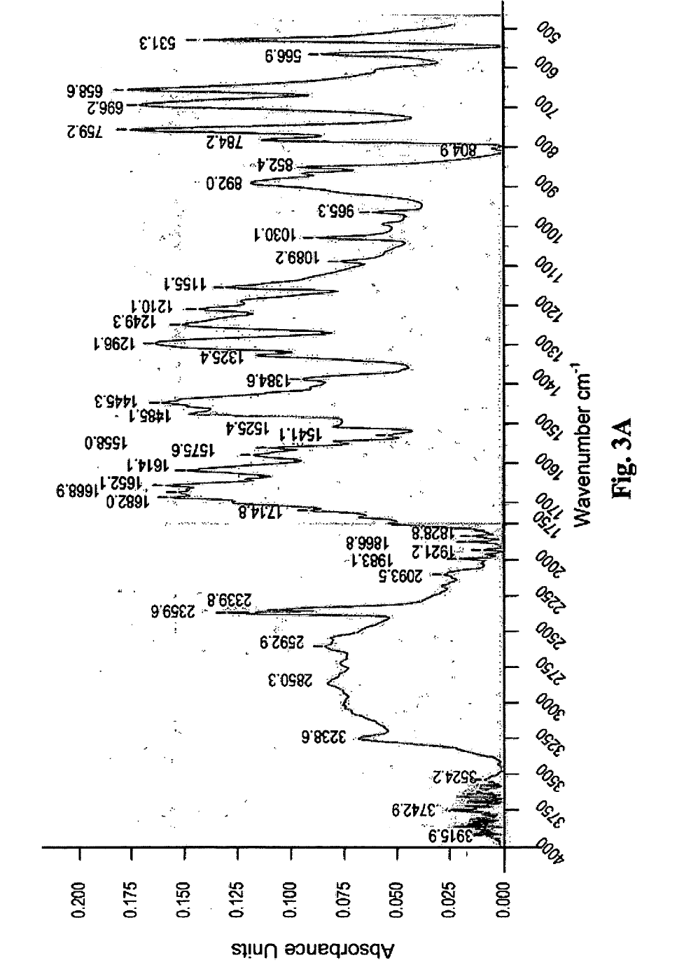

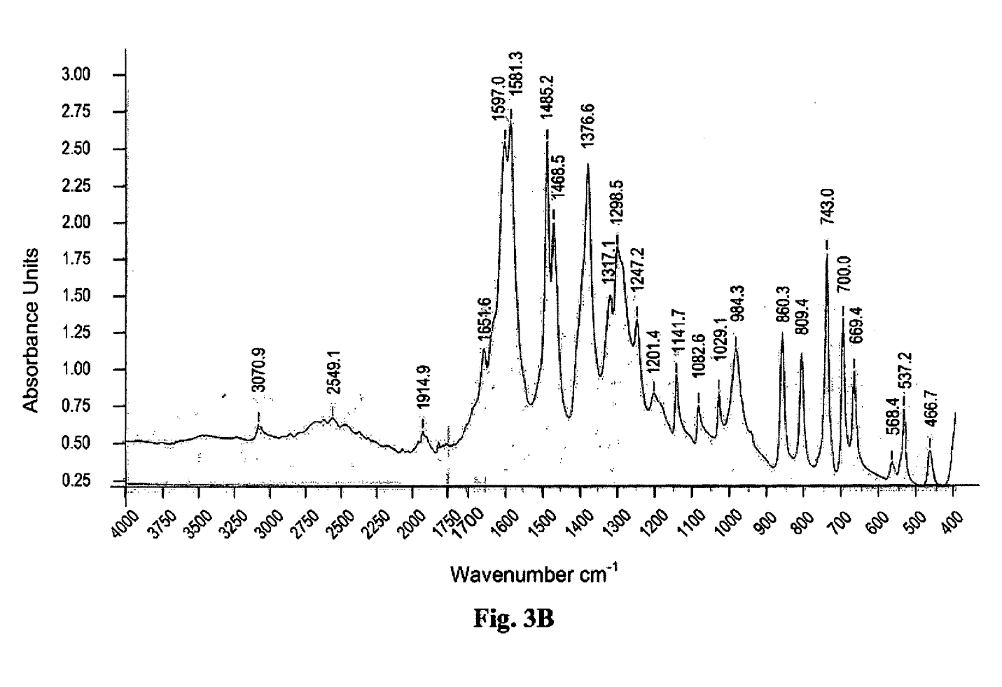

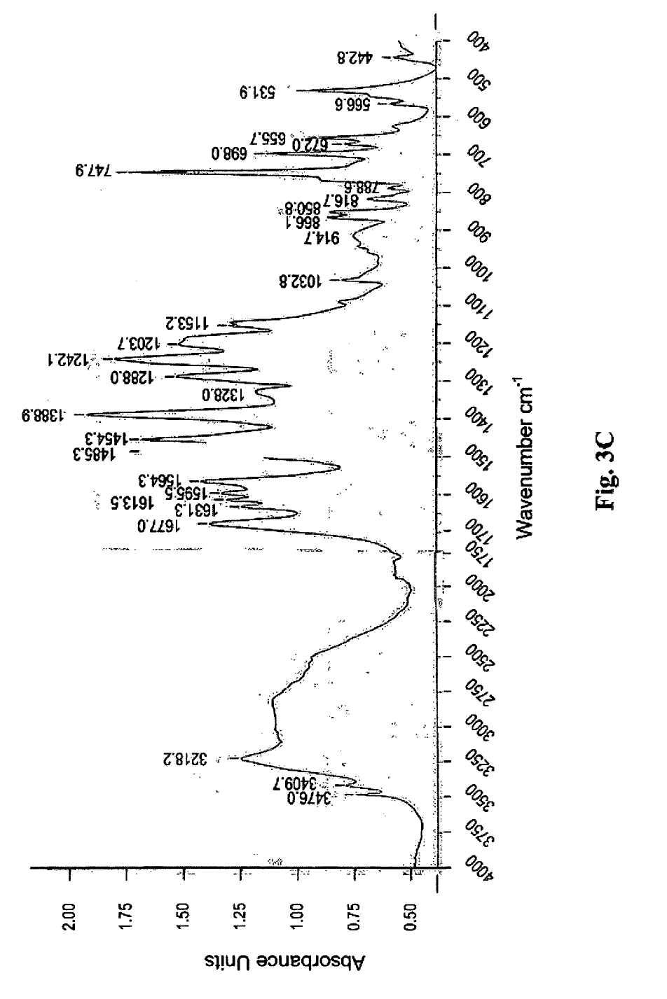

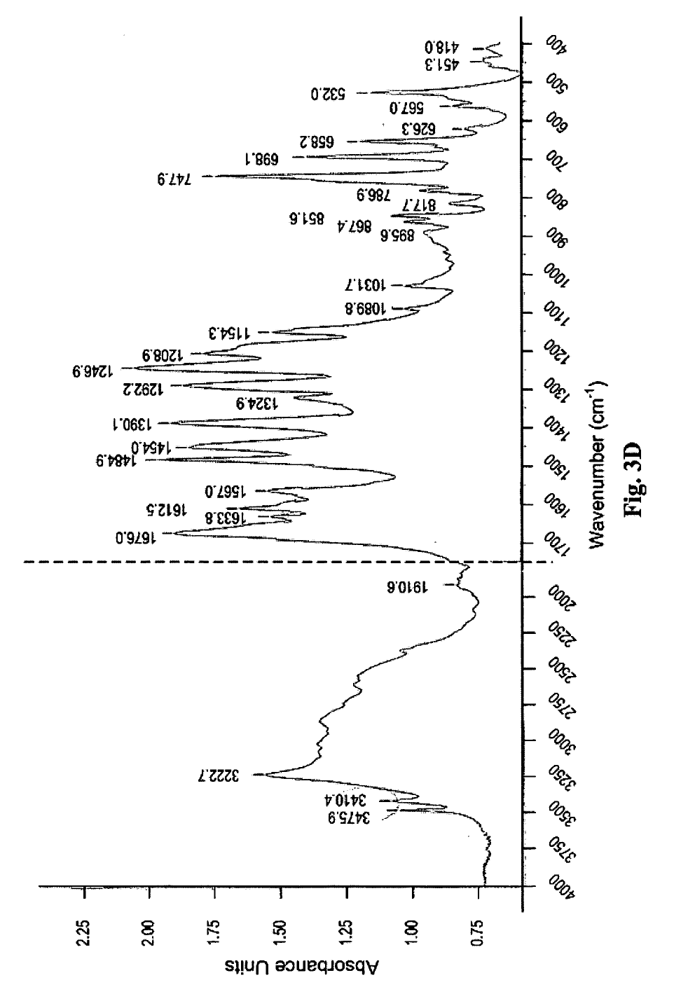

Fourier transform infrared (FTIR) spectroscopy analysis was performed for inclusion complexes of salicylic acid (7%) which were prepared by the two-step (35-57-2, prepared with 0.18% polyacrylamide and 2% urea) and one-step (35-87-2, prepared with 0.2% polyacrylamide and 2.1% urea) organic solvent-free processes. The results are shown in FIGS. 3A-3D, in which FIG. 3A depicts the infrared analysis of pure salicylic acid, FIG. 3B depicts the infrared analysis of the sodium salicylate salt (prepared by mixing salicylic acid with an approximately equimolar amount of NaOH, final pH 10), and FIGS. 3C-3D depict the absorbance profiles of salicylic acid nanoparticles prepared by the two-step process (sample 35-57-2) and the one-step process, respectively, It is to be noted that FIG. 3C and FIG. 3D are essentially identical. A summary comparison of the outstanding points of these absorbance profiles is presented in Table 4. The peak at 1581.3, that is unique for the salt, can be attributed to carboxylate anion stretching. The observation that this peak is not found in the spectra of the nanoparticles indicates that these inclusion complexes are not salicylic acid salts. Additionally, there is a focused peak at 1676.6, that is associated with the inclusion complexes and is more diffuse for pure salicylic acid. This may be the result of directed carbonyl stretching in the complexes.

TABLE 4
FTIR analysis of salicylic acid (SA),
salicylate salt and SA nano-particles

| | Wavelength (cm⁻¹) | | | |
|---|---|---|---|---|
| Sample | 2500-3500 | 1676.6 | 1581.3 | 1454 |
| SA | + | + | − | + |
| | | (1682-1652) | | (1485) |
| Sodium salicylate salt | − | − | + | − |
| SA nano-particles | + | + | − | + |
| (2-step process*) | | | | |
| SA nano-particles | + | + | − | + |
| (1-step process*) | | | | |

*7% SA dispersions prepared with 0.33% polyacrylamide modified with 2% urea

### Example 5

### Release of Salicylic Acid from Nanoparticles Comprising Inclusion Complexes of Salicylic Acid

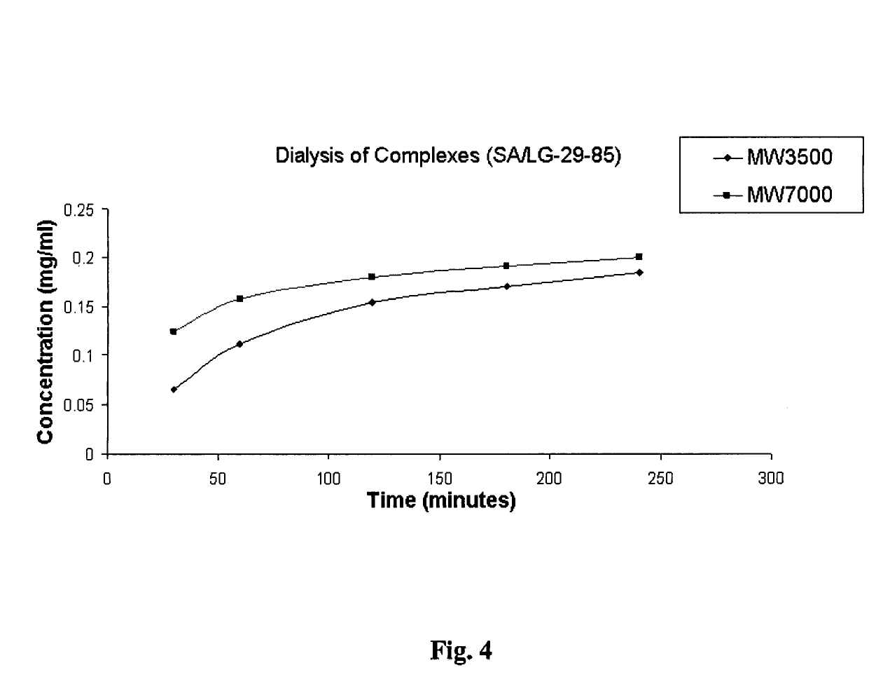

Release of salicylic acid from the inclusion complexes was assessed in vitro by monitoring changes in the salicylic acid concentration following salicylic acid passage through dialysis tubing. Dialysis tubing (Spectra/Por) having a pore size of either 3500 Daltons or 7000 Daltons was used, since the polymer is significantly greater than 7000 Daltons. The tubing was filled with 2 ml of a dispersion of nanoparticles (sample SA/LG-29-85, prepared using the two-step method with 1% or 2% urea and 0.2% PAA concentration, as described in Example 2) having a final salicylic acid concentration of 7%. The filled tubing was suspended in a beaker that contained 100 ml of alcohol (external solution). The external solution was continuously stirred in order to maintain a homogeneous salicylic acid concentration. At the times indicated in FIG. 4, 1 ml aliquots were removed from the external solution for analysis of the salicylic acid content by reverse-phase high pressure liquid chromatography (RP-HPLC). This analysis entailed preparation of a standard salicylic acid curve in which there was a linear relation between the salicylic acid concentration and the area of the measured salicylic acid samples of the curve. Measurement of the salicylic acid in each experimental sample was followed by calculation of the salicylic acid concentration according to the area of salicylic acid peak in that sample.

The results in FIG. 4 demonstrate that different rates of release were obtained when dialysis tubing with different pore sizes was used. The initial dialysis rate was faster when the pore size was larger. However, by four hours, the salicylic acid concentration in the external solution was similar in both experiments such that approximately 14% of the salicylic acid had migrated through the membrane. At this time point, in both cases, the salicylic acid concentration is still one tenth of its normal maximal solubility. Thus, the inclusion complexes provide a system that modifies salicylic acid release.

### Example 6

### Preparation of Polymers—Modified Starch

(i) Hydrolyzed Potato Starch (HPS) 3.8% with H₂O₂ (1°)—Polymer A

Polymer A was prepared by adding 20 g potato starch to 500 ml of water, adding 0.2 ml of 20% citric acid and mixing. Autoclaving was carried out for 60 min (1.58-1.61 atm, 113-115° C.). Hydrogen peroxide was added (15 ml 33% H₂O₂) at temperature 67° C. under mixing with magnet stirrer for 60 minutes. After cooling to room temperature, pH, turbidity and viscosity of the solution were measured. The values obtained were: pH 3.5±0.4, turbidity 33±2 FTU (formazin turbidity unit), and viscosity 20±2 cP (centipoises).

In this and in the following examples, turbidity was measured with a SMART 2 colorimeter (LaMotte Company, Chestertown, Mass., USA), using the turbidity mode for this measurement; viscosity was measured with Visco Star Plus (measurements were made at a room temperature, spindle TL5, 100 rpm).

(ii) Modified Food (corn) Starch B-790 (Pure-Cote B-790®, Grain Processing Corp., Muscatine, Iowa, USA) 3.8%—Polymer B

Polymer B was prepared by adding 24 g starch B-790 to 600 ml of water under mixing with magnetic stirrer and heating at 70-80° C. for 180±10 min. After cooling to room temperature, the mixture was filtered through the filter paper MN 615¼, and pH, turbidity and viscosity of the solution were measured. The values obtained were: pH 5.5±0.3, turbidity 200±10 FTU and viscosity 10±2 cP.

### Example 7

### Preparation of Solu-Sodium Hyaluronate (Solu-NaHA), 0.1%

Preparation of 0.2% solutions of sodium hyaluronate of two different molecular weights (NaHA; MW 3 million Da and 1.3 million Da, NaHA from human umbilical cord, SIGMA, H 1876) was carried out by dissolution of 0.2 g of NaHA in 100 ml water at room temperature with mixing on magnet stirrer without heating during 120±10 min.

NaCl was added to the final concentration of 1.7% (w/w): 1.7 g NaCl to 100 ml 0.2% solution of NaHA, and mixed for 5-10 min. 50 ml of Polymer A or Polymer B were placed in a three-necked flask of 150 ml and heated in a water-bath up to the temperature 54-56° C. An equal volume (50 ml) of 0.2% NaHA solution was added dropwise to the polymer solution (0.35 ml in 1 minute) with constant mixing by a mechanical glass stirrer utilizing a teflon tip (stirring rate —300 rpm). Upon completion, the solution was cooled under constant mixing at 30-32° C. The final product, herein designated Solu-NaHA, is an opalescent solution, concentration of NaHA—0.1%.

The pH, viscosity and size of the particles were measured. Viscosity was measured by Visco Star Plus (Fungilab SA, Spain) at room temperature; size of particles was measured by dynamic light scattering with a Malvern Zeta Sizer. The values obtained were: pH 4.0±0.5, viscosity 13±2 cP. The average particle diameter of the Solu-NaHA was 100-130 nm.

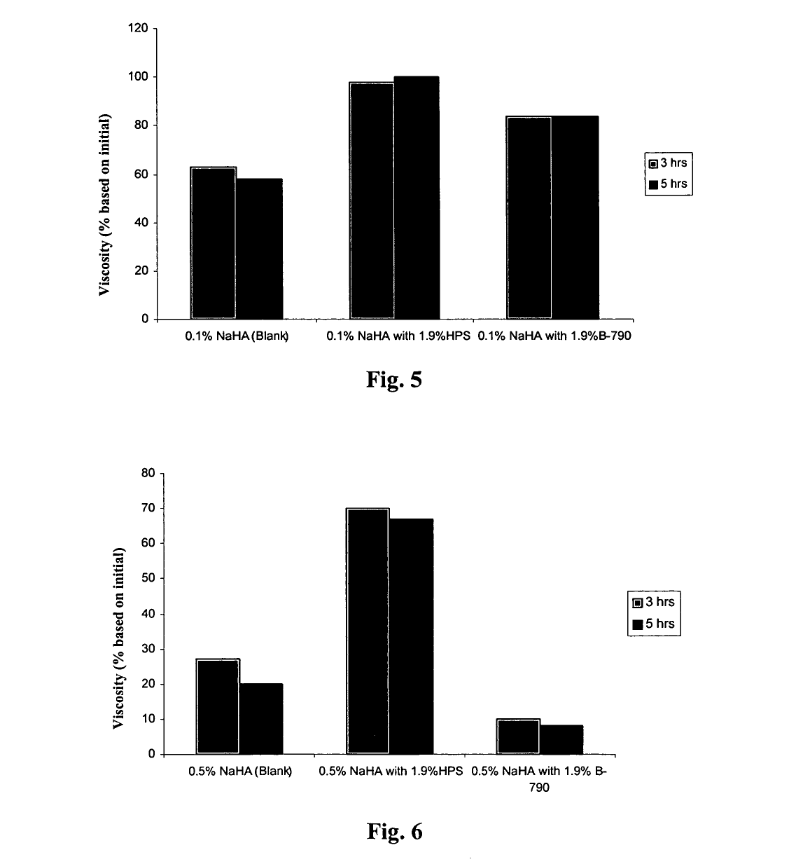

The stability of Solu-NaHA in the presence of the enzyme specific for hyaluronic acid, hyaluronidase (Sigma, H 3506, Hyaluronidase lyophilized (EC 3.2.1.35) Type I-S, from bovine testes, 608 U/mg solid) was measured by the decrease of the viscosity in time in comparison to blank. The degree of stability (protection against action of hyaluronidase) is defined as a decrease in the viscosity of the NaHA solution upon addition of the enzyme (dose of enzyme—10 U/ml). The control used was 0.1% solution of NaHA without the wrapping polymer (blank). Samples were maintained on a water bath at temperature 37° C. during 5 hrs. Sample made by Visco Star Plus. The decrease in viscosity was estimated in percentage to its initial value. The results in FIG. 5 show the stability of Solu-NaHA prepared with polymer A (NaHA with HPS) or with Polymer B (NaHA with B-790) against the action of hyaluronidase, established as 84-100% vs control 58-63%.

### Example 8

### Preparation of Sodium Hyaluronate (Solu-NaHA), 0.5%

Preparation of 1% solutions of sodium hyaluronate of two different molecular weights [NaHA; MW 3 million Da and 1.3 million Da, Sigma, H 1876, Hyaluronic acid sodium salt from human umbilical cord] was carried out by dissolution of 10 g of NaHA in 100 ml water at room temperature with mixing on magnet stirrer without heating during 300±30 min.

NaCl was added to the final concentration of 1.7% (w/w): 1.7 g NaCl to 100 ml 1.0% solution of NaHA, and mixed for 5-10 min. 50 ml of Polymer A or Polymer B were placed in a three-necked flask of 150 ml and heated in a water-bath up to the temperature 54-56° C. An equal volume (50 ml) of 1.0% NaHA solution was added dropwise to the polymer solution (0.35 ml in 1 minute) with constant mixing by a mechanical glass stirrer utilizing a teflon tip (stirring rate: 300 rpm). Upon completion, the solution was cooled under constant mixing at 30-32° C. The final product, herein designated Solu-NaHA, is an opalescent solution, concentration of NaHA-0.5%.

The pH, viscosity and size of the particles were measured. The values obtained were: pH 4.0±0.5, viscosity 50±10 cP. The average particle diameter of the Solu-NaHA was 100-140 nm.

The stability of NaHA in the Solu-NaHA in the presence of hyaluronidase was measured by the decrease of the viscosity over time in comparison to blank. The results are shown in FIG. 6. The stability of Solu-NaHA against the action of hyaluronidase was 70-90% vs 40-45% in blank (0.5% solution of NaHA).

The degree of stability of NaHA was measured as described in Example 7 above. The control used was 0.5% solution of NaHA without any polymer (blank). Samples were maintained on a water bath at temperature 37° C. during 5 hrs. Measurement of viscosity was made by Visco Star Plus. Decrease in viscosity is estimated in percentage relative to its initial value. It is established, that protection of Solu-NaHA with polymer A (HPS) against action of hyaluronidase gives a viscosity of 67-70% vs the control protection of 20-27%. However, Solu-NaHA with polymer B (B-790) does not show such activity and its viscosity under enzyme activity decreases to 8-10% vs the control 20-27%. Hence, polymer B is not effective for obtaining Solu-NaHA which demonstrates enhanced protection against enzymatic degradation.

### Example 9

### Preparation of Solu-Bovine Serum Albumin (BSA), 2.4%

Preparation of 4.8% solutions of bovine serum albumin (BSA) was carried out by dissolution of 5.0 g of BSA (Merck, K 31587018 320, Albumin from bovine serum, Fraction Y) in 100 ml water at room temperature with mixing on magnet stirrer without heating during 10 min.

NaCl was added to the final concentration of 1.7% (w/w): 1.7 g NaCl to 100 ml 4.8% solution of BSA, and mixed for 5-10 min. 50 ml of Polymer B were placed in a three-necked flask of 150 ml in a water-bath at room temperature (no heating is used for proteins). An equal volume (50 ml) of 4.8% BSA solution was added dropwise to the polymer solution (0.35 ml in 1 minute) with constant mixing by a mechanical glass stirrer utilizing a teflon tip (stirring rate: 300 rpm). The final product, herein designated Solu-BSA, is an opalescent solution, concentration of BSA—2.4%.

The pH, viscosity, size of the particles, and stability under acidic conditions (pH 1.5) were measured. The values obtained were: pH 6.5±0.4, viscosity 11±2 cP. The average particle diameter of the Solu-SBA was 90-120 nm. The stability under acidic conditions was at least for 1.5 hours.

Stability of Solu-BSA under acidic conditions is estimated based on checking for changes in the particle size: absence of change indicates stability. Continuous particle size measurements were made using Malvern light diffraction instrumentation during at least 1.5 hours at temperature 25° C. During this time the disperse system of Solu-BSA remained stable, the average size of particles did not vary.

### Example 10

### Preparation of Solu-Hyaluronidase (Solu-Hd), 0.2%

Preparation of 0.4 solutions of hyaluronidase (Hd) (Sigma, H 3506, Hyaluronidase lyophilized (EC 3.2.1.35) Type I-S, from bovine testes, 608 U/mg solid) was carried out by dissolution of 0.4 g of Hd in 100 ml water at room temperature with mixing on magnet stirrer without heating during 10 min.

NaCl was added to the final concentration of 1.7% (w/w): 1.7 g NaCl to 100 ml 0.4% solution of Hd, and mixed for 5-10 min. 50 ml of Polymer B were placed in a three-necked flask of 150 ml in a water-bath at room temperature. An equal volume (50 ml) of 0.4% Hd solution was added dropwise to the polymer solution (0.35 ml in 1 minute) with constant mixing by a mechanical glass stirrer utilizing a teflon tip (stirring rate: 300 rpm). The final product, herein designated Solu-Hd, is an opalescent solution, concentration of Hd—0.2%.

The pH, viscosity, size of the particles, and stability under acidic conditions (pH 1.5) were measured. The values obtained were: pH 5.0±0.2, viscosity 10±2 cP. The average particle diameter of the Solu-Hd was 150-200 nm. The stability under acidic conditions was at least for 1.5 hours.

Stability is checked by looking changes in particle size. Continuous measurements using Malvern light diffraction instrumentation for at least 1.5 hours at temperature 25° C. were conducted. During this time the disperse system of Solu-Hd remained stable, the average size of particles did not vary.

### Example 11

### Preparation of Nanoparticles of Inclusion Complexes of Azithromycin Wrapped in Chitosan—SoluAzi A

This experiment was carried out in 3 steps:

(i) Step 1

Chitosan (Kraeber GmbH & Co, Germany; pharmaceutical grade, dynamic viscosity 430 mPas (millipascal seconds)) was added to a 1% acetic acid solution (1 g chitosan, 100 ml water, 1 ml acetic acid 99%), and the mixture was heated to 65-70° C. At the end of the dissolution process (about 60-90 minutes), the resulting polymer solution contained some undissolved residue and had a pH of 4.0-4.4 and viscosity 80-100 cP.

In order to remove these residues, a process using high water volumes, heat and intensive homogenization was employed. Thus, water was first added to the polymer solution such that the solution volume increased 3-4 times as compared to the original volume. Afterward, the solution was heated at 65-70° C. while applying high speed homogenization (˜10,000 rpm). This process continued at a rate of 10 ml/min, and was followed by filtration of this mixture (using suitable filters with pore size between 0.3-0.5 mm). The filtrate was then concentrated (evaporated) till it reached its initial concentration. (The whole procedure of chitosan dissolution in water was performed in vessel 1). The resultant polymer solution had the following characteristics: pH=4.0-4.4, viscosity 25-30 cP, conductivity S=1.24-1.64 ms/cm (millisiemens/cm). Viscosity was measured with Visco Star Plus (spindle TL5, 100 rpm) and conductivity was measured with a conductivity meter (Jenco Electronics Ltd., Model 1671).

ii. Step 2

Azithromycin (Assia Chemical Ind., Israel) powder was added to the acidic chitosan aqueous solution in vessel 1 while mixing with a magnetic stirrer (500-1000 rpm) at 65-70° C. such as to obtain a 1% solution (w/v) of azithromycin in the chitosan aqueous solution. This process continued until all the drug has dissolved completely.

iii. Step 3

In this step, the azithromycin/chitosan aqueous solution and a large volume of water (3-4 times the volume of the drug/polymer solution) contained in a vessel 2 were simultaneously streamed to a high turbulent zone of a vessel 3 equipped with a high speed homogenizer (above 10,000 rpm) for interaction of the polymer with azithromycin and formation of the SoluAzi solumer. The water was streamed at a higher speed than the azithromycin/chitosan solution. The azithromycin/chitosan solution was then transferred to a vessel 4 and was heated steadily at 65-70° C. and stirred wiyh with a magnetic stirrer (500-1000 rpm). At the end of this process, the vessel 4 contained all the SoluAzi that was formed having the original concentration (1% azithromycin in chitosan solution). The final product SoluAzi A had the following characteristics: 1% azithromyzin; 1% chitosan; viscosity 25 cP; pH=5.6-5.7; conductivity S=1.65 ms/cm and particles sizes 600-700 nm.

Using this technology, solumers were obtained containing 2% and 4% azithromycin. These solumers had the following characteristics:

4% SoluAzi Solumer: 4% azithromyzin; 1.4% chitosan; viscosity 60 cP; pH 6.16; conductivity S=4.74 ms/cm; particles size 500-700 nm.

2% SoluAzi Solumer: 2% azithromyzin; 2% chitosan; viscosity 25 cP; pH 6.2; conductivity S=3.1 ms/cm; particles size 500-700 nm.

### Example 12

### Preparation of Nanoparticles of Inclusion Complexes of Azithromycin Wrapped in Chitosan—SoluAzi B

In this procedure, chitosan (Chimarin™, Medicarb, Sweden) of significantly lower viscosity (22 mPas) was used. Due to this amenable viscosity of this range, no prior treatment of the polymer was required. The experiment was carried out in 3 steps:

(i) Step 1

For the preparation of an aqueous solution of chitosan, 1% chitosan (viscosity 22 mPas) was added to 0.6% acetic acid solution while heating at a temperature of 65-70° C. The time of dissolution was 60 minutes. When the chitosan was dissolved, the polymer solution had the following characteristics: viscosity 23 cP; pH=4.62; and conductivity, S=2.4 ms/cm.

Steps 2 and 3 were carried out as described in Example 11 above. A solumer was obtained with the following characteristics: azithromyzin 1%; chitosan 1%; viscosity 11 cP; pH=5.6; conductivity, S=1.6 ms/cm; and particles size 500-700 nm.

1. A solvent-free process for the preparation of a hydrophilic dispersion comprising nanoparticles of an hydrophilic inclusion complex consisting essentially of nanosized particles of an active compound and an amphiphilic polymer which wraps said active compound such that non-valent bonds are formed between said compound and said polymer in said inclusion complex, comprising:
(i) preparation of an aqueous solution of the amphiphilic polymer; and
(ii) bringing the active compound and the polymer aqueous solution into interaction under conditions suitable for the formation of said hydrophilic dispersion.

2. The process according to claim 1, wherein in step (ii) the active compound is added as a powder to the polymer aqueous solution.

3. The process according to claim 1, wherein in step (ii) an aqueous solution of the active compound is added dropwise to the polymer aqueous solution under constant mixing.

4. The process according to claim 1, wherein the active compound is a small organic molecule.

5. The process according to claim 1, wherein the active compound is a macromolecule.

6. The process according to claim 2, for the preparation of a hydrophilic dispersion comprising nanoparticles of inclusion complexes of salicylic acid wrapped in an amphiphilic polymer such that non-valent bonds are formed between the salicylic acid and the amphiphilic polymer, wherein said amphiphilic polymer is selected from the group consisting of polyacrylic acid, polyacrylamide and copolymers thereof, polymethacrylamide and copolymers thereof, and polylysine, and said polymer is modified by reaction with urea or a derivative thereof, nicotinamide or guanidine, comprising:
(i) preparation of a solution of the amphiphilic polymer in water;
(ii) modification of the amphiphilic polymer by reaction with urea or a derivative thereof, nicotinamide or guanidine, under heat and pressure, in an autoclave;
(iii) addition of salicylic acid powder to the modified polymer water solution; and
(iv) subjecting the dispersion obtained in (iii) to autoclave treatment, thus obtaining the desired dispersion comprising nanoparticles of inclusion complexes of salicylic acid entrapped within said modified amphiphilic polymer.

7. The process according to claim 6, wherein the amphiphilic polymer is selected from the group consisting of polyacrylamide, polymethacrylamide and a copolymer of acrylamide or methacrylamide with one or two monomers selected from the group consisting of acrylic acid, methacrylic acid, an alkyl acrylate, an alkyl methacrylate, acrylonitrile, ethyleneimine, vinyl acetate, styrene, maleic anhydride and vinyl pyrrolidone, and the polymer modifier is urea or a urea derivative selected from the group consisting of methylol urea, acetyl urea, semicarbazide and thiosemicarbazide.

8. The process according to claim 7, wherein the amphiphilic polymer is polyacrylamide modified by reaction with urea.

9. The process according to claim 2, for the preparation of a hydrophilic dispersion comprising nanoparticles of inclusion complexes of salicylic acid wrapped in an amphiphilic polymer such that non-valent bonds are formed between the salicylic acid and the amphiphilic polymer, wherein said amphiphilic polymer is selected from the group consisting of polyacrylic acid, polyacrylamide and copolymers thereof, polymethacrylamide and copolymers thereof, and polylysine, and said polymer is modified by reaction with urea or a derivative thereof, nicotinamide or guanidine, comprising:
(ii) preparation of a solution of the amphiphilic polymer in water;
(ii) modification of the amphiphilic polymer by reaction with urea or a derivative thereof, nicotinamide or guanidine;
(iii) addition of salicylic acid powder to the modified polymer water solution; and
(iv) subjecting the dispersion obtained in (iii) to autoclave treatment, thus obtaining the desired hydrophilic dispersion comprising nanoparticles of inclusion complexes of salicylic acid entrapped within said amphiphilic polymer.

10. The process according to claim 9, wherein the amphiphilic polymer is selected from the group consisting of polyacrylamide, polymethacrylamide and a copolymer of acrylamide or methacrylamide with one or two monomers selected from the group consisting of acrylic acid, methacrylic acid, an alkyl acrylate, an alkyl methacrylate, acrylonitrile, ethyleneimine, vinyl acetate, styrene, maleic anhydride and vinyl pyrrolidone, and the polymer modifier is urea or a urea derivative selected from the group consisting of methylol urea, acetyl urea, semicarbazide and thiosemicarbazide.

11. The process according to claim 10, wherein the amphiphilic polymer is polyacrylamide modified by reaction with urea.

12. The process according to claim 3, for the preparation of a hydrophilic dispersion comprising nanoparticles of inclusion complexes of an active macromolecule and an amphiphilic polysaccharide which wraps the active macromolecule such that non-valent bonds are formed between said active macromolecule and said amphiphilic polysaccharide, the process comprising the steps of:
(i) preparing a solution of the amphiphilic polysaccharide in water;
(ii) preparing a molecular solution of the active macromolecule in water and
(iii) adding dropwise the water solution of the active macromolecule (ii) into the water polysaccharide solution (i) under constant mixing;
thus obtaining the hydrophilic dispersion comprising nanoparticles of inclusion complexes of said active macromolecule wrapped within said amphiphilic polysaccharide.

13. The process according to claim 12, wherein said active macromolecule is a naturally-occurring, synthetic or recombinant polypeptide of molecular weight above 1,000 Da, protein, nucleic acid or polysaccharide, and the amphiphilic polysaccharide is natural or modified starch, chitosan or alginate.

14. The process according to claim 12, wherein the polysaccharide is potato, maize/corn, wheat, or tapioca/cassava starch modified in order to increase its hydrophilicity, to reduce the amount of branching, or both.

15. The process according to claim 13, wherein said polypeptide or protein is a naturally-occurring, synthetic or recombinant hormone, cytokine or chemokine, enzyme, immunoglobulin or monoclonal antibody.

16. The process according to claim 15, wherein said polypeptide or protein is added to the polysaccharide solution in step (iii) at room temperature.

17. The process according to claim 16, wherein the protein is hyaluronidase.

18. The process according to claim 11, wherein the active macromolecule is a polysaccharide selected from the group consisting of hyaluronic acid and its salts, chondroitin sulphate, dermatin sulphate, heparan sulphate, lentinan, and heparin and its derivatives including low molecular weight heparins (LMWH).

19. The process according to claim 18, wherein said polysaccharide is added to the warmed polysaccharide solution in step (iii).

20. The process according to claim 18, wherein said polysaccharide is hyaluronic acid or a salt thereof.

21. The process according to claim 3, process for the preparation of a hydrophilic dispersion comprising nanoparticles of an hydrophilic inclusion complex consisting essentially of nanosized particles of a macrolide antibiotic and an amphiphilic polysaccharide which wraps said active compound such that non-valent bonds are formed between said macrolide antibiotic and said polysaccharide in said inclusion complex, comprising:
(i) preparation of an acidic aqueous solution of the amphiphilic polysaccharide;
(ii) adding the macrolide antibiotic powder to the acidic aqueous polymer solution of (i) under heating and mixing for dissolution of the macrolide antibiotic;
(iii) streaming the solution of (ii) simultaneously with a large volume of water to a high turbulent zone of a vessel for the interaction of the macrolide antibiotic with the polysaccharide; and
(iv) concentrating the aqueous solution of (iii) under constant mixing and heating,
thus obtaining the desired dispersion of nanoparticles of inclusion complexes of the macrolide antibiotic wrapped in the polysaccharide.

22. The process according to claim 21, wherein said macrolide antibiotic is erythromycin, clarithromycin or azithromycin, and the amphiphilic polysaccharide is natural or modified starch, chitosan or alginate.

23. The process according to claim 22, wherein the macrolide antibiotic is azithromycin and the polysaccharide is chitosan.
